(19) United States
(12) Patent Application Publication
(10) Pub. No.: US 2019/0110780 A1
(43) Pub. Date: Apr. 18, 2019

(54) Height-Adjustable Skin Excision Biopsy Device

(71) Applicant: Nami Bayan, Shelton, CT (US)

(72) Inventor: Nami Bayan, Shelton, CT (US)

(21) Appl. No.: 15/988,556

(22) Filed: May 24, 2018

Related U.S. Application Data
(60) Provisional application No. 62/571,980, filed on Oct. 13, 2017.

(51) Int. Cl. A61B 10/02; A61B 17/3205

(52) U.S. Cl. CPC A61B 10/0266; A61B 2017/00367; A61B 2010/0208; A61B 17/3205

(57) ABSTRACT

A height-adjustable skin excision biopsy device consists of a hollow cylindrical handle, an outer hub, a flexible slider bar, an inner hub, an elliptical blade section, a height-adjusting mechanism, and a sliding mechanism. The flexible slider bar is terminally and perpendicularly connected to the inner hub. The hollow cylindrical handle is perpendicularly and terminally connected to the outer hub. The flexible slider bar is slidably positioned within the hollow cylindrical handle so that the inner hub is sleeved by the outer hub. In other words, the flexible slider bar can vertically move within the hollow cylindrical handle through the height-adjusting mechanism. Moreover, the inner hub can slide along the outer hub through the sliding mechanism. The elliptical blade section is connected to the inner hub opposite the flexible slider bar. Therefore, the position of the elliptical blade section can be controlled through the flexible slider bar.

The current application claims a priority to the U.S. Provisional Patent application Ser. No. 62/571,980 filed on Oct. 13, 2017.

### FIELD OF THE INVENTION

The present invention relates generally to a medical device used for excision of skin lesions. More specifically, the present invention is a height-adjustable lesion excision device intended to accelerate the process, increase efficiency and increase accuracy of skin lesion excision biopsies.

### BACKGROUND OF THE INVENTION

Excision biopsy of skin lesions are performed by qualified medical professionals to cut through the skin of patients and remove suspicious malignant or premalignant lesions.

The excision biopsy procedure must be carried out in a very cautious manner to minimize procedure related complications, and to reach the desirable outcomes in the best way possible. However, due to a variety of reasons the excision biopsies might result in unwanted and inappropriate consequences due to flaws related to the procedure technique, instruments and human errors.

In general, prior to a skin excision biopsy, an elliptical area surrounding the lesion is initially marked by using a ruler and a marking pen. Next, the skin is locally anesthetized with lidocaine or similar topical anesthetic. Then, with the use of a scalpel knife, the excision is executed along the area that is marked out on the skin. The process might not only increase the time of procedure, but also lead to technical flaws, errors in measurements, visual misperception, miscalculation in estimation, and flaws due to dexterity issues of the operating medical professional. Thus, the incision can have a serrated edge, various depths and less than perfect dimensions. Consequently, these flaws may increase the risk of complications, and reduce the quality of wound healing and increase the scarring.

Since no additional device is used during the excision, the accuracy of the procedure relies on the skill level of the individual executing the procedure. As an example, if the medical professional is not experienced, the probability of error can be high. Similarly, in another instance, if the medical professional has less than ideal hand eye coordination, the probability of error can be higher. Also, visual misperception, miscalculation in measurements, and imperfect cuts by the scalpel can increase the chance of complications and unwanted outcomes. When using the scalpel, even experienced medical professionals can make errors leading to imperfect incisions.

The objective of the present invention is to address the aforementioned issues. In particular, the present invention intends to make the excision procedure more convenient, more accurate and of higher quality. The present invention is used as a guide so that the excision procedure is fast and has a perfect incision which does not vary from one medical professional to another. Moreover, the height adjustability of the present invention allows the operator to optimize the depth of the incision based on skin thickness and the depth of tissue needed to be removed.

### BRIEF DESCRIPTION OF THE DRAWINGS

FIG. 1A is a perspective view of the present invention.

FIG. 1B is a perspective view of the present invention, wherein the imprinting blade cover is illustrated.

FIG. 2 is another perspective view of the present invention, wherein the elliptical blade section is extended through the height-adjusting mechanism.

FIG. 3 is a side view of the present invention.

FIG. 4 is another side view of the present invention, wherein the height-adjusting mechanism and the sliding mechanism are illustrated.

FIG. 5 is a front view of the present invention.

FIG. 6 is a top view of the present invention.

FIG. 7 is a bottom view of the present invention.

FIG. 8 is a perspective exploded view of the outer hub, the inner hub, and the elliptical blade section.

FIG. 9 is a perspective view of the outer hub.

FIG. 10 is a perspective view of the inner hub and the elliptical blade section.

### DETAIL DESCRIPTIONS OF THE INVENTION

All illustrations of the drawings are for the purpose of describing selected versions of the present invention and are not intended to limit the scope of the present invention.

The present invention is a medical device that is used to execute skin excision biopsies. More specifically, the present invention standardizes skin biopsies and does so with greater convenience and higher accuracy levels.

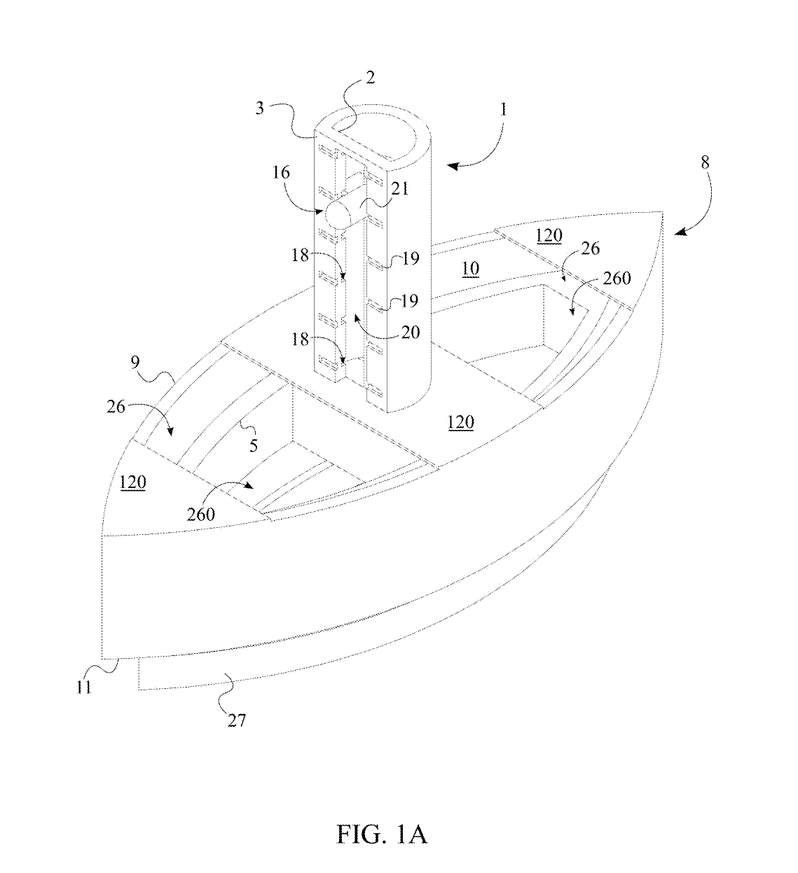

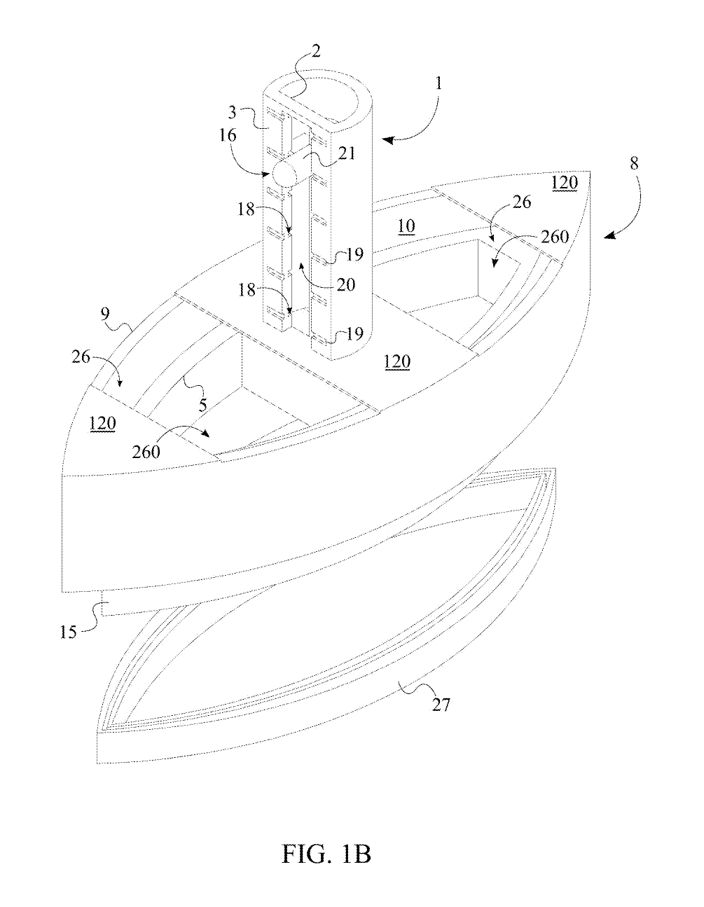

As illustrated in FIGS. 1A-4, the present invention comprises a hollow cylindrical handle 1, an inner hub 4, an outer hub 8, a flexible slider bar 13, an elliptical blade section 15, a height-adjusting mechanism 16, and a sliding mechanism 22. The flexible slider bar 13, made of a flexible but sturdy polymer, is used to control a blade height of the elliptical blade section 15. To do so, the flexible slider bar 13 is perpendicularly and terminally connected to a top surface 12 of the inner hub 4. To be controlled by the flexible slider bar 13, the elliptical blade section 15 is terminally connected to the inner hub 4 opposite the flexible slider bar 13. Since the flexible slider bar 13, the inner hub 4, and the elliptical blade section 15 are interconnected, the position of the elliptical blade section 15 can be controlled by moving the flexible slider bar 13 in a vertical direction. To move the flexible slider bar 13 in a vertical direction, and thus control the blade height of the elliptical blade section 15, the flexible slider bar 13 is slidably positioned within the hollow cylindrical handle 1 through the height-adjusting mechanism 16. To be positioned around the flexible slider bar 13, and to be held by the medical professional during the excision procedure, the hollow cylindrical handle 1 is perpendicularly and terminally connected to a top surface 120 of the outer hub 8. The inner hub 4 is sleeved by the outer hub 8 so that the elliptical blade section 15 protrudes out from the outer hub 8 according to the blade height selected by the user. As an example, if the blade height is selected to be 3-millimeters (mm), and the overall height of the elliptical blade section 15 is 6-mm, 3-mm of the elliptical blade section 15 will protrude out from the outer hub 8. The remaining 3-mm of the elliptical blade section 15 will be concealed within the outer hub 8. To be exposed as required, the inner hub 4 is slidably positioned within the outer hub 8 through the sliding mechanism 22.

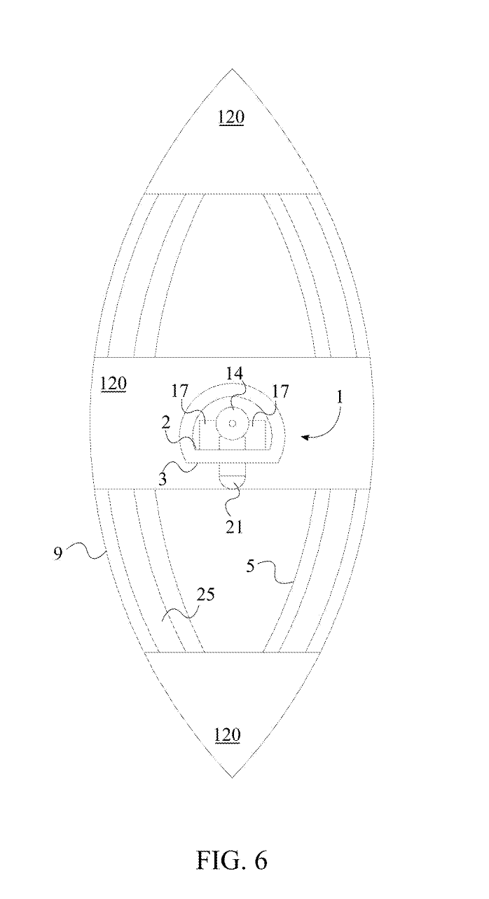

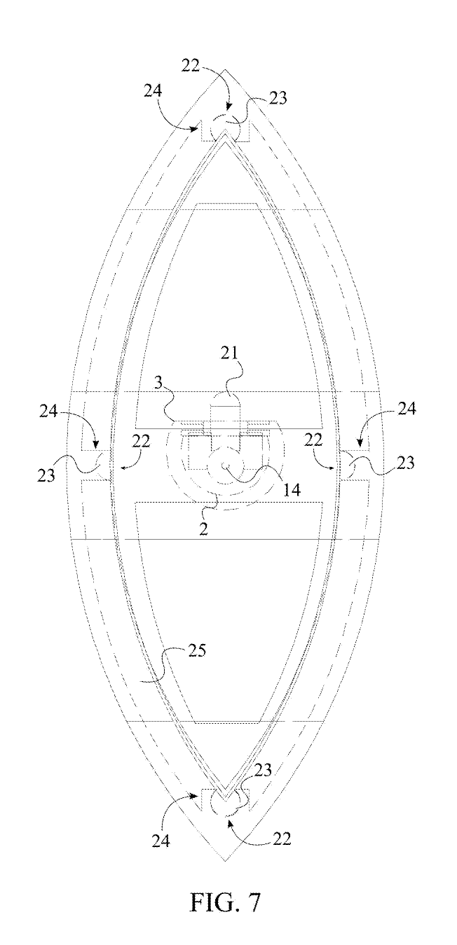

As discussed before, the elliptical blade section 15 is terminally connected to the inner hub 4. As illustrated in FIGS. 6-10, to accommodate the elliptical shape of the elliptical blade section 15, the inner hub 4 is also elliptical in shape. The inner hub 4 is sleeved by the outer hub 8. Therefore, to accommodate the elliptical shape of the inner hub 4 the outer hub 8 is also elliptical in shape. In the preferred embodiment of the present invention, a ratio between a minor axis of the elliptical blade section 15 and a major axis of the elliptical blade section 15 is 1:3.3. The elliptical blade section 15 can vary in overall size as long as the 1:3.3 ratio remains unchanged. For instance, in one embodiment, the minor axis can be 3-mm so that the major axis is 10-mm. In another embodiment, the minor axis can be 30-mm so that the major axis is 99-mm. The different sizes allow the user to select the most appropriate size based upon the lesion, the possible pathology of the skin lesion, and the location of the lesion. The material used to manufacture the elliptical blade section 15 can vary in different embodiments of the present invention. In the preferred embodiment, the elliptical blade section 15 is made of stainless steel. However, other comparable materials can be used in other embodiments of the present invention. Even though an elliptical shape is described for the elliptical blade section 15, the inner hub 4, and the outer hub 8, the overall shape can be described as a keel shape or canoe shape. As seen in FIG. 6 and FIG. 7, to obtain the canoe shape, the elliptical blade section 15, the inner hub 4, and the outer hub 8 will have angulated or narrow ends.

In the preferred embodiment of the present invention, the inner hub 4 and the outer hub 8 are made of a transparent polymer. Thus, the medical professional can view the elliptical blade section 15 being in contact with the skin of the patient during the excision procedure. Since the lesion can be viewed through the inner hub 4 and the outer hub 8, inaccuracies related to the excision procedure are eliminated.

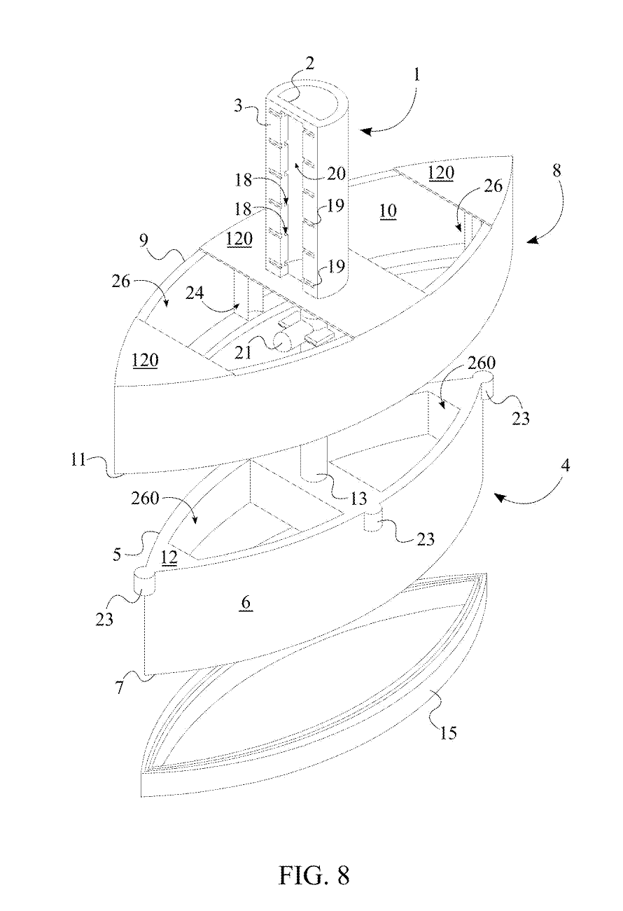

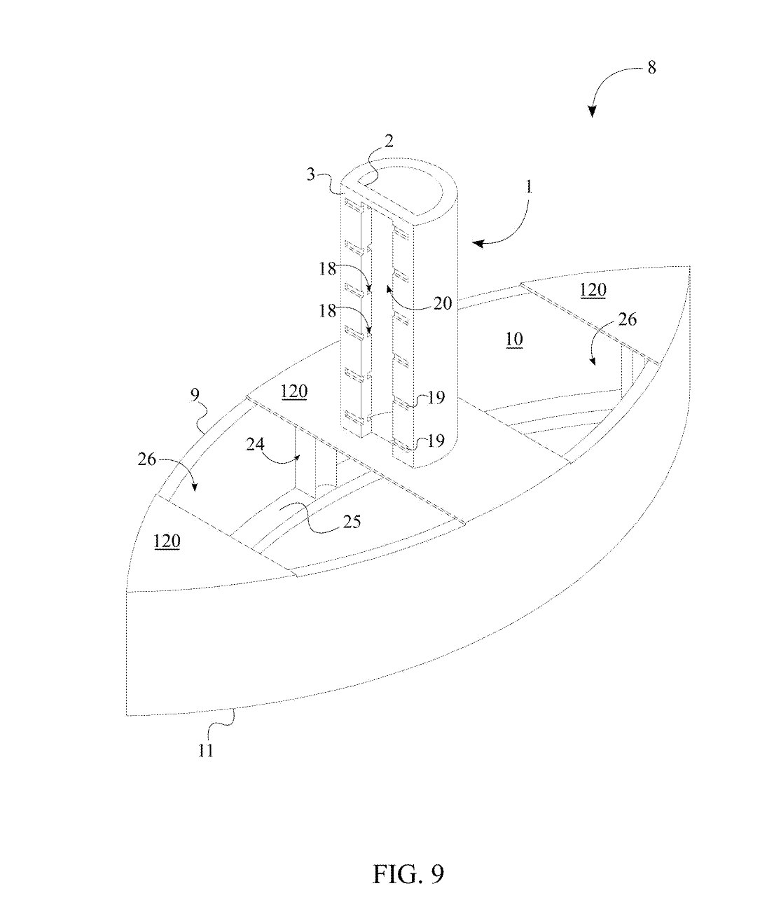

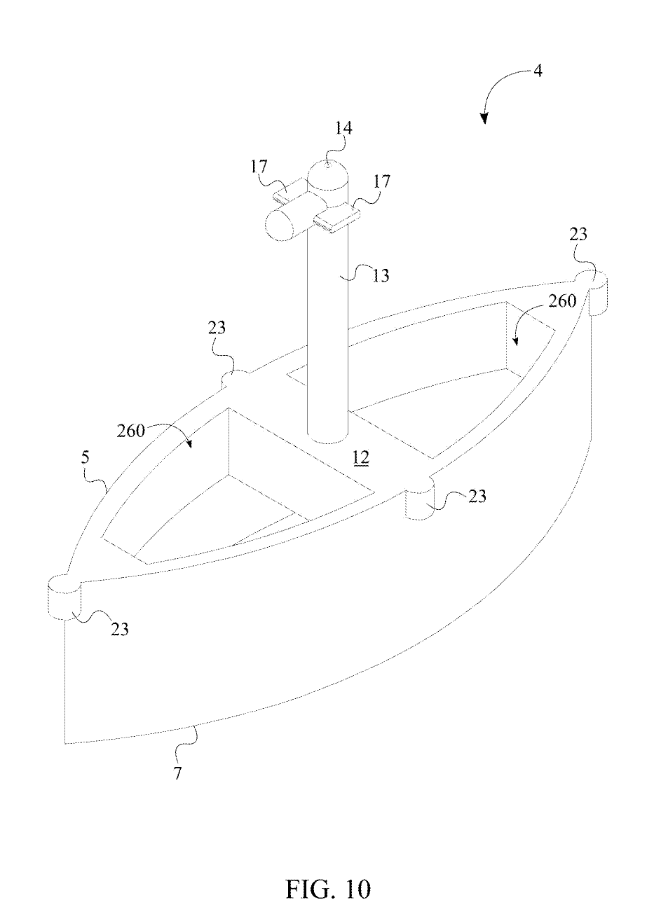

As described, the elliptical blade section 15 is terminally and perimetrically connected to the inner hub 4. Thus, when viewing the elliptical blade section 15 at an angle through the top surface 12 of the inner hub 4 and the top surface 120 of the outer hub 8, refraction can occur and mislead the user. As shown in FIG. 8 and FIG. 9, to address the issue, the present invention further comprises at least one first viewing window 26 that perpendicularly traverses into the top surface 120 of the outer hub 8. Since the hollow cylindrical handle 1 is perpendicularly and terminally connected to the top surface 120 of the outer hub 8, the at least one first viewing window 26 is positioned adjacent the hollow cylindrical handle 1. In addition to minimizing refraction, the at least one first viewing window 26 allows light to reach the lesion and the surrounding skin. As seen in FIG. 10, to transfer the external light through the inner hub 4 and onto the skin of the patient, the inner hub 4 further comprises at least one second viewing window 260 that perpendicularly traverses through the top surface 12 of the inner hub 4. Since the flexible slider bar 13 is perpendicularly and terminally connected to the top surface 12 of the inner hub 4, the at least one second viewing window 260 is positioned adjacent the flexible slider bar 13.

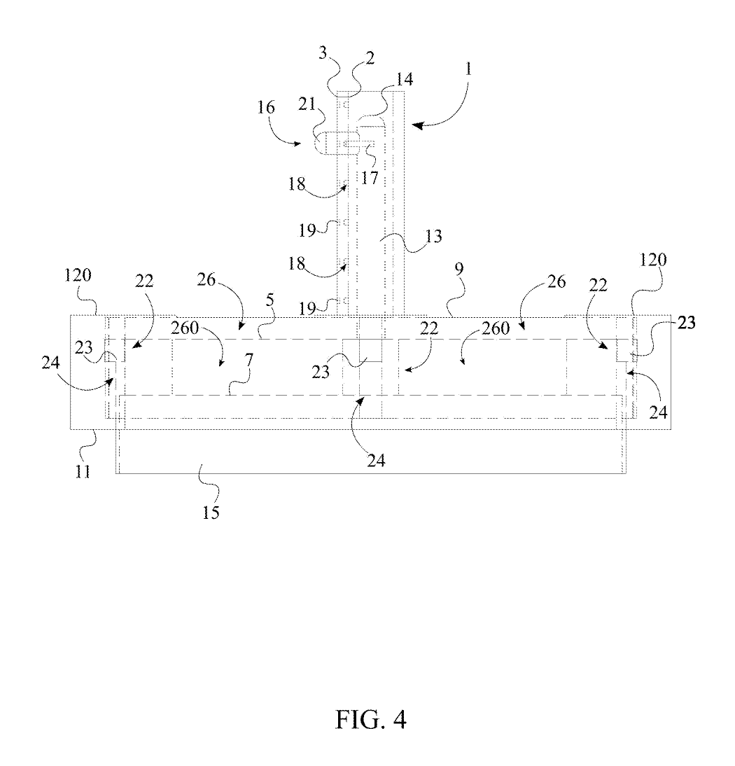

As mentioned earlier, the height-adjusting mechanism 16 allows the flexible slider bar 13 to be positioned at a preferred height within the hollow cylindrical handle 1. In the preferred embodiment, the height-adjusting mechanism 16 comprises a pair of engaging teeth 17 and a plurality of teeth engaging slots 18. As shown in FIG. 10, each of the pair of engaging teeth 17 is laterally and perpendicularly connected to the flexible slider bar 13. As shown in FIG. 9, to receive the pair of engaging teeth 17, the plurality of teeth engaging slots 18 is vertically distributed along an inner surface 2 of the hollow cylindrical handle 1. When the preferred height is selected by the user, the pair of engaging teeth 17 is removably positioned into a corresponding slot from the plurality of teeth engaging slots 18 as seen in FIG. 4. To do so, the user pushes on the flexible slider bar 13, moves the flexible slider bar 13 in the vertical direction to the preferred height, and releases the flexible slider bar 13. The flexibility allows the flexible slider bar 13 to recoil so that the pair of engaging teeth 17 is positioned into the corresponding slot from the plurality of teeth engaging slots 18 as seen in FIG. 4.

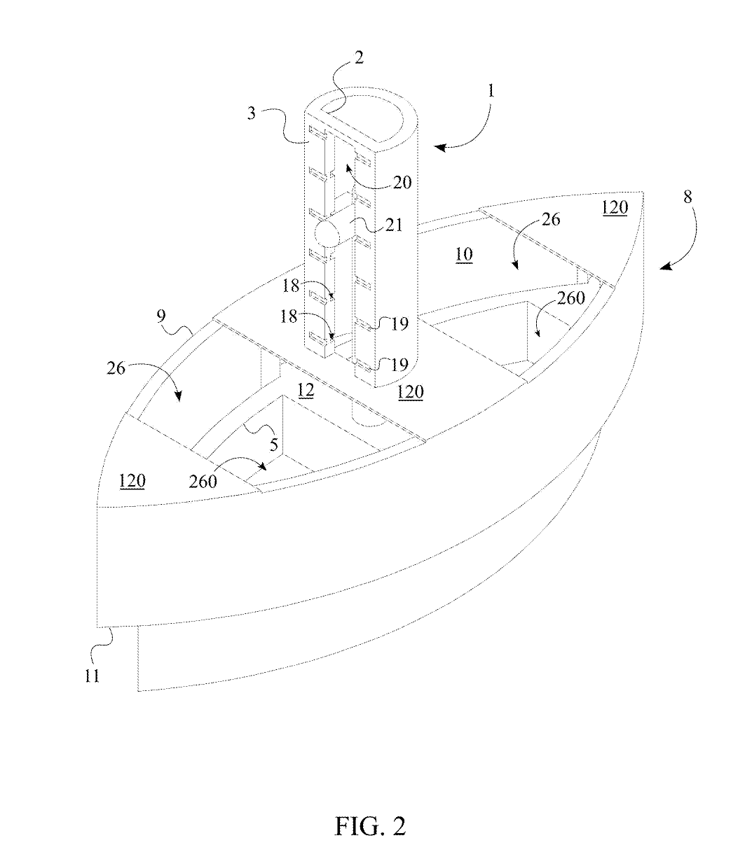

As seen in FIG. 1A and FIG. 2, when the user moves the flexible slider bar 13 in a vertical direction within the hollow cylindrical handle 1, the inner hub 4 and the elliptical blade section 15 that is connected to the inner hub 4 also moves in the corresponding vertical direction. To do so, the inner hub 4 is slidably positioned within the outer hub 8 through the sliding mechanism 22. The sliding mechanism 22 can vary in different embodiments of the present invention. As seen in FIGS. 7-9, in the preferred embodiment, the sliding mechanism 22 comprises a plurality of sliding tabs 23, a plurality of guide rails 24, and a stoppage tab 25. The plurality of sliding tabs 23 is connected to the inner hub 4 and the plurality of guide rails 24 is connected to the outer hub 8. Thus, by positioning the plurality of sliding tabs 23 into the plurality of guide rails 24, the inner hub 4 can slide along the outer hub 8 according to the vertical movement of the flexible slider bar 13.

As shown in FIG. 8, to position the plurality of sliding tabs 23, the inner hub 4 comprises a top edge 5, an external lateral wall 6, and a bottom edge 7. The external lateral wall 6 extends from the top edge 5 to the bottom edge 7. The plurality of sliding tabs 23 is radially and equidistantly mounted onto the external lateral wall 6 adjacent the top edge 5. The number of sliding tabs used as the plurality of sliding tabs 23 can vary from one embodiment to another. In the preferred embodiment, four sliding tabs are used.

As seen in FIG. 8 and FIG. 9, to receive the plurality of sliding tabs 23 and to position the plurality of guide rails 24, the outer hub 8 comprises an upper lip 9, an internal lateral wall 10, and a lower lip 11. The internal lateral wall 10 extends from the upper lip 9 to the lower lip 11. Since the inner hub 4 is sleeved by the outer hub 8, the external lateral wall 6 is positioned adjacent and perimetrically around the internal lateral wall 10. The plurality of guide rails 24 is radially and equidistantly positioned along the internal lateral wall 10 and extends from the upper lip 9 to the lower lip 11. For the inner hub 4 to be slidably positioned with the outer hub 8, each of the plurality of sliding tabs 23 is slidably positioned into a corresponding rail from the plurality of guide rails 24. Since the plurality of guide rails 24 extends from the upper lip 9 to the lower lip 11, the plurality of sliding tabs 23 can slide from the upper lip 9 to the lower lip 11. The stoppage tab 25 is perimetrically positioned along the internal lateral wall 10 adjacent the lower lip 11 so that the plurality of sliding tabs 23 is halted at the lower lip 11.

In reference to FIG. 1A and FIG. 2, when the flexible slider bar 13 is moved in a vertical direction, the interconnection through the inner hub 4 makes the elliptical blade section 15 move in the corresponding vertical direction. The vertical movement of the inner hub 4 in conjunction with the positioning of the inner hub 4 within the outer hub 8, determines the blade height of the elliptical blade section 15. As an example, when the top edge 5 of the inner hub 4 is positioned adjacent the upper lip 9 of the outer hub 8, the elliptical blade section 15 is exposed minimally. On the other hand, when the top edge 5 of the inner hub 4 is pressed against the stoppage tab 25 at the lower lip 11 of the outer hub 8, the elliptical blade section 15 is completely exposed.

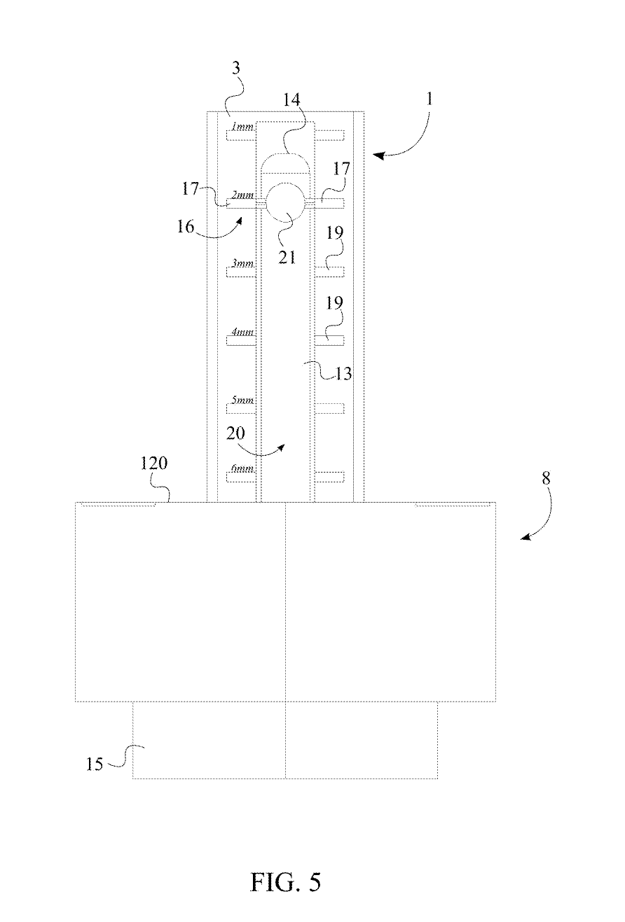

As shown in FIG. 5, to determine the exact blade height of the elliptical blade section 15, the height-adjusting mechanism 16 further comprises a plurality of indices 19 that is vertically distributed along an outer surface 3 of the hollow cylindrical handle 1. Each of the plurality of indices 19 is aligned with a corresponding slot from the plurality of teeth engaging slots 18. As a result, when the flexible slider bar 13 is positioned at a preferred height through the height-adjusting mechanism 16, the plurality of indices 19 shows a numerical value for the preferred height. In the preferred embodiment of the present invention, a first index of the plurality of indices 19 is 1-mm and corresponds to a topmost slot from the plurality of teeth engaging slots 18. On the other hand, a last index of the plurality of indices 19 is 6-mm and corresponds to a bottommost slot from the plurality of teeth engaging slots 18. However, the range of the plurality of indices 19 can vary in other embodiments of the present invention.

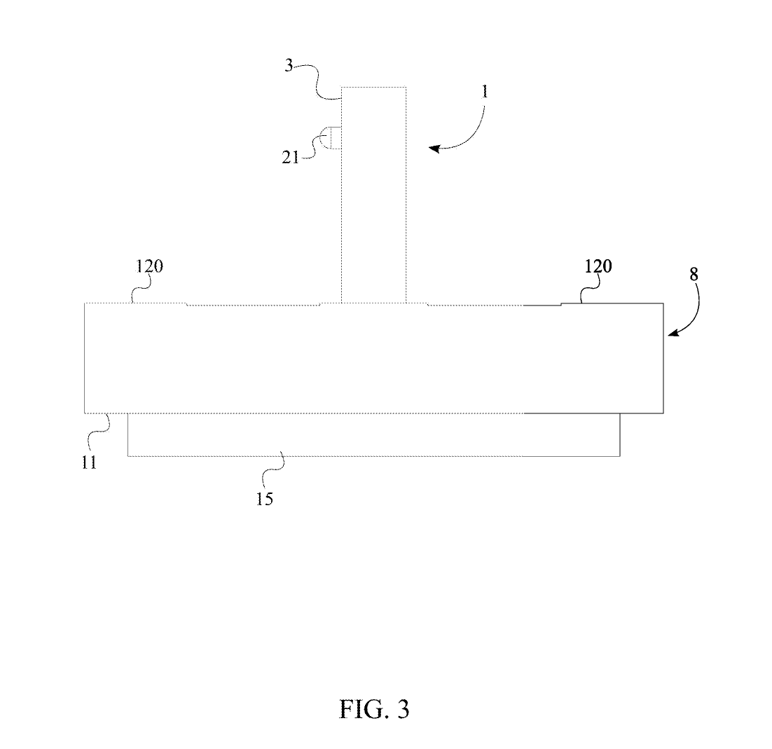

As seen in FIG. 3 and FIG. 4, the present invention further comprises a push tab 21 that serves as an extension from the flexible slider bar 13 positioned within the hollow cylindrical handle 1. To control the flexible slider bar 13, the push tab 21 is perpendicularly and terminally mounted to the flexible slider bar 13 adjacent a top end 14 of the flexible slider bar 13. In the preferred embodiment, the push tab 21 is positioned in between each of the pair of engaging teeth 17. As seen in FIG. 5, to position the push tab 21 in an accessible position, the height-adjusting mechanism 16 further comprises an elongated slit 20 that traverses into the hollow cylindrical handle 1. The push tab 21 extending from the flexible slider bar 13 is positioned through the elongated slit 20. Since the elongated slit 20 is positioned adjacent the plurality of teeth engaging slots 18 and adjacent the plurality of indices 19, the user can utilize the push tab 21, the plurality of indices 19, and the plurality of teeth engaging slots 18 simultaneously.

As an example, if the flexible slider bar 13 is positioned at 1-mm on the plurality of indices 19, the pair of engaging teeth 17 will be positioned into the corresponding slot from the plurality of teeth engaging slots 18 denoted 1-mm. Moreover, 1-mm of the elliptical blade section 15 will extend out from the lower lip 11 of the outer hub 8. In another instance, if the flexible slider bar 13 is positioned at 5-mm on the plurality of indices 19, 5-mm of the elliptical blade section 15 will extend from the lower lip 11 of the outer hub 8. In general, the height selected through the height-adjusting mechanism 16 determines the blade height of the elliptical blade section 15 which then ultimately determines the depth of the incision.

With existing methods, the skin surrounding the lesion is marked with the use of a ruler and a pen. Since no guide is used in the marking process, the probability of error is significant. As shown in FIG. 1A and FIG. 1B, to fulfill the need of outlining the lesion, the present invention further comprises an imprinting blade cover 27 that is removably attached to the elliptical blade section 15 and has an overall elliptical shape. When attached to the elliptical blade section 15, the imprinting blade cover 27 is positioned opposite to the inner hub 4 so that the imprinting blade cover 27 can be conveniently accessed. The imprinting blade cover 27 stamps a guide line around the lesion prior to the excision procedure. For imprinting purposes, the imprinting blade cover 27 can be layered with ink or other comparable material. Prior to the excision procedure, the imprinting blade cover 27 is removed so that the elliptical blade section 15 is revealed.

When the present invention is being used, the following process flow is generally followed. Initially, the skin lesion is roughly measured. Based on the size of the skin lesion, location of the skin lesion, the possible skin pathology, required safety margin, and tension lines, the appropriate size of the elliptical blade section 15 that needs to be used is determined.

As an example, if the patient is suspected of having melanoma, a 5-mm safety margin needs to be on each side of the lesion to assure the removal of malignant cells. If the lesion is approximately 10-mm in length, the user selects the present invention such that the minor axis of the elliptical blade section 15 is 20-mm and the major axis of the elliptical blade section 15 is 66-mm. In another instance, if the patient is suspected to have basal cell carcinoma, a 1-mm or a 2-mm safety margin is required. If the approximate length of the lesion is 10-mm, the user selects the present invention such that the minor axis of the elliptical blade section 15 is 12-mm and the major axis of the elliptical blade section 15 is 40-mm.

When the appropriate size is determined, the medical professional proceeds with the necessary prep and drape. Next, the skin around the lesion is marked out with the use of the imprinting blade cover 27. When the guide line is established around the lesion, topical anesthetic is applied to the marked skin area around the lesion. Based upon the location of the lesion, the medical professional determines the required height of the elliptical blade section 15. As an example, if the lesion is on the face of the patient, a minimal height of the elliptical blade section 15 is used. On the other hand, if the lesion is on the foot of the patient, an ideal height is selected for the elliptical blade section 15.

To obtain the preferred height for the elliptical blade section 15, the user pushes the push tab 21 so that the pair of engaging teeth 17 disengages from the corresponding slot on the plurality of teeth engaging slots 18. Thus, the flexible slider bar 13 can freely move in a vertical direction. Consequently, the height of the elliptical blade section 15 can be altered. When the preferred height is obtained by moving the flexible slider bar 13 in the vertical direction, the push tab 21 is released so that the pair of engaging teeth 17 is positioned into a new slot of the plurality of teeth engaging slots 18. The new slot corresponds to the preferred height of the elliptical blade section 15. The preferred height can be accurately determined by utilizing the plurality of indices 19 since each of the plurality of indices 19 is aligned with each of the plurality of teeth engaging slots 18.

After the skin of the patient is prepared for the excision procedure and the preferred height is selected for the elliptical blade section 15, the present invention is placed over the lesion such that the elliptical blade section 15 is positioned along the previously established guide line. When positioned appropriately, the lesion is viewed through the at least one first viewing window 26 and the at least one second viewing window 260. Since the inner hub 4 and the outer hub 8 are transparent, the lesion can be clearly viewed. Next, the medical professional applies gentle pressure perpendicular to the skin by holding the hollow cylindrical handle 1. As a result, an incision of desired depth is made. As an example, we will consider the need for an incision with a depth of 4-mm. When the flexible slider bar 13 is positioned appropriately, 4-mm of the elliptical blade section 15 protrudes from the lower lip 11 of the outer hub 8. Thus, when the user presses on the skin, the present invention makes a cut with a depth of 4-mm.

By utilizing the present invention, the overall procedure time for a biopsy is minimized. For instance, the need to precisely measure the lesion, and the need to mark the skin with a ruler and a pen are eliminated with the use of the present invention. Moreover, the present invention replaces the time-consuming process of making an incision with a scalpel.

The effective design of the present invention, eliminates or minimizes human error, instrument errors, errors due to dexterity, and errors due to visual and sensory misperception. Moreover, the present invention allows a user of any experience level to utilize the present invention.

An incision made with a scalpel can lead to many unfavorable circumstances such a serrated wound edge, soft tissue damage, uneven incision depth, and subcutaneous vessel damage. By utilizing the present invention, a uniform incision is made through the elliptical blade section 15 by gently applying pressure perpendicular to the skin of the patient. The uniform incision helps the wound heal better with less scarring.

Although the invention has been explained in relation to its preferred embodiment, it is to be understood that many other possible modifications and variations can be made without departing from the spirit and scope of the invention as hereinafter claimed.

What is claimed is:

1. A height-adjustable skin excision biopsy device comprises:
a hollow cylindrical handle;
an inner hub;
an outer hub;
a flexible slider bar;
an elliptical blade section;
a height-adjusting mechanism;
a sliding mechanism;
the flexible slider bar being perpendicularly and terminally connected to a top surface of the inner hub;
the elliptical blade section being terminally connected to the inner hub, opposite the flexible slider bar;
the hollow cylindrical handle being perpendicularly and terminally connected to a top surface of the outer hub;
the flexible slider bar being slidably positioned within the hollow cylindrical handle through the height-adjusting mechanism;
the inner hub being sleeved by the outer hub; and
the inner hub being slidably positioned within the outer hub through the sliding mechanism.

2. The height-adjustable skin excision biopsy device as claimed in claim 1, wherein both the inner hub and the outer hub are elliptical in shape.

3. The height-adjustable skin excision biopsy device as claimed in claim 1, wherein both the inner hub and the outer hub are made of a transparent polymer.

4. The height-adjustable skin excision biopsy device as claimed in claim 1 further comprises:
the outer hub further comprises at least one first viewing window;
the at least one first viewing window perpendicularly traversing into a top surface of the outer hub; and
the at least one first viewing window being positioned adjacent the hollow cylindrical handle.

5. The height-adjustable skin excision biopsy device as claimed in claim 1 further comprises:
the inner hub comprises at least one second viewing window;
the at least one second viewing window perpendicularly traversing into the top surface of the inner hub; and
the at least one second viewing window being positioned adjacent the flexible slider bar.

6. The height-adjustable skin excision biopsy device as claimed in claim 1 further comprises:
the sliding mechanism comprises a plurality of sliding tabs, a plurality of guide rails, and a stoppage tab;
the inner hub comprises a top edge, an external lateral wall, and a bottom edge;
the outer hub comprises an upper lip, an internal lateral wall, and a lower lip;
the external lateral wall extending from the top edge to the bottom edge;
the internal lateral wall extending from the upper lip to the lower lip;
the stoppage tab being perimetrically positioned along the internal lateral wall, adjacent the lower lip;
the plurality of sliding tabs being radially and equidistantly mounted onto the external lateral wall, adjacent the top edge;
the plurality of guide rails being radially and equidistantly positioned along the internal lateral wall;
the plurality of guide rails extending from the upper lip to the lower lip; and
each of the plurality of sliding tabs being slidably positioned into a corresponding rail from the plurality of guide rails.

7. The height-adjustable skin excision biopsy device as claimed in claim 1 further comprises:
the height-adjusting mechanism comprises a pair of engaging teeth and a plurality of teeth engaging slots;
the hollow cylindrical handle comprises an inner surface;
the plurality of teeth engaging slots being vertically distributed along the inner surface;
each of the pair of engaging teeth being laterally and perpendicularly connected to the flexible slider bar; and
the pair of engaging teeth being removably positioned into a corresponding slot from the plurality of teeth engaging slots.

8. The height-adjustable skin excision biopsy device as claimed in claim 7 further comprises:
the height-adjusting mechanism further comprises a plurality of indices;
the hollow cylindrical handle further comprises an outer surface;
the plurality of indices being vertically distributed along the outer surface opposite to the plurality of teeth engaging slots; and
each of the plurality of indices being aligned with a corresponding slot from the plurality of teeth engaging slots.

9. The height-adjustable skin excision biopsy device as claimed in claim 8, wherein a first index of the plurality of indices is 1 millimeter.

10. The height-adjustable skin excision biopsy device as claimed in claim 8, wherein a final index of the plurality of indices is 6 millimeters.

11. The height-adjustable skin excision biopsy device as claimed in claim 1 further comprises:
a push tab;
the height-adjusting mechanism further comprises an elongated slit;
the flexible slider bar comprises a top end;
the elongated slit traversing through the hollow cylindrical handle;
the elongated slit being positioned adjacent the plurality of teeth engaging slots;
the push tab being perpendicularly and terminally mounted onto the flexible slider bar adjacent the top end; and
the push tab being positioned through the elongated slit.

12. The height-adjustable skin excision biopsy device as claimed in claim 1 further comprises:
an imprinting blade cover; and
the imprinting blade cover being removably attached to the elliptical blade section opposite to the inner hub.

13. A height-adjustable skin excision biopsy device comprises:
a hollow cylindrical handle;
an inner hub;
an outer hub;
a flexible slider bar;
an elliptical blade section;
a height-adjusting mechanism;
a sliding mechanism;
an imprinting blade cover;
the outer hub further comprises at least one first viewing window;
the inner hub comprises at least one second viewing window;
the flexible slider bar being perpendicularly and terminally connected to a top surface of the inner hub;
the elliptical blade section being terminally connected to the inner hub, opposite the flexible slider bar;
the hollow cylindrical handle being perpendicularly and terminally connected to a top surface of the outer hub;
the flexible slider bar being slidably positioned within the hollow cylindrical handle through the height-adjusting mechanism;
the inner hub being sleeved by the outer hub;
the inner hub being slidably positioned within the outer hub through the sliding mechanism;
the at least one first viewing window perpendicularly traversing into a top surface of the outer hub;
the at least one first viewing window being positioned adjacent the hollow cylindrical handle;
the at least one second viewing window perpendicularly traversing into the top surface of the inner hub;
the at least one second viewing window being positioned adjacent the flexible slider bar; and
the imprinting blade cover being removably attached to the elliptical blade section opposite to the inner hub.

14. The height-adjustable skin excision biopsy device as claimed in claim 13, wherein both the inner hub and the outer hub are elliptical in shape.

15. The height-adjustable skin excision biopsy device as claimed in claim 13, wherein both the inner hub and the outer hub are made of a transparent polymer.

16. The height-adjustable skin excision biopsy device as claimed in claim 13 further comprises:
the sliding mechanism comprises a plurality of sliding tabs, a plurality of guide rails, and a stoppage tab;
the inner hub comprises a top edge, an external lateral wall, and a bottom edge;
the outer hub comprises an upper lip, an internal lateral wall, and a lower lip;
the external lateral wall extending from the top edge to the bottom edge;
the internal lateral wall extending from the upper lip to the lower lip;
the stoppage tab being perimetrically positioned along the internal lateral wall, adjacent the lower lip;
the plurality of sliding tabs being radially and equidistantly mounted onto the external lateral wall, adjacent the top edge;
the plurality of guide rails being radially and equidistantly positioned along the internal lateral wall;
the plurality of guide rails extending from the upper lip to the lower lip; and
each of the plurality of sliding tabs being slidably positioned into a corresponding rail from the plurality of guide rails.

17. The height-adjustable skin excision biopsy device as claimed in claim 13 further comprises:
the height-adjusting mechanism comprises a pair of engaging teeth, a plurality of teeth engaging slots, and a plurality of indices;
the hollow cylindrical handle comprises an inner surface and an outer surface;
the plurality of teeth engaging slots being vertically distributed along the inner surface;
each of the pair of engaging teeth being laterally and perpendicularly connected to the flexible slider bar;
the pair of engaging teeth being removably positioned into a corresponding slot from the plurality of teeth engaging slots;
the plurality of indices being vertically distributed along the outer surface opposite to the plurality of teeth engaging slots; and
each of the plurality of indices being aligned with a corresponding slot from the plurality of teeth engaging slots.

18. The height-adjustable skin excision biopsy device as claimed in claim 17, wherein a first index of the plurality of indices is 1 millimeter.

19. The height-adjustable skin excision biopsy device as claimed in claim 17, wherein a final index of the plurality of indices is 6 millimeters.

20. The height-adjustable skin excision biopsy device as claimed in claim 13 further comprises:
a push tab;
the height-adjusting mechanism further comprises an elongated slit;
the flexible slider bar comprises a top end;
the elongated slit traversing through the hollow cylindrical handle;
the elongated slit being positioned adjacent the plurality of teeth engaging slots;
the push tab being perpendicularly and terminally mounted onto the flexible slider bar adjacent the top end; and
the push tab being positioned through the elongated slit.
